(19) United States
(12) Patent Application Publication
(10) Pub. No.: US 2009/0012324 A1
(43) Pub. Date: Jan. 8, 2009

(54) Process for Production of Carboxylic Acid Ester or Ether Compound

(76) Inventors: Jun-Chul Choi, Ibaraki (JP); Kazufumi Kohno, Ibaraki (JP); Hiroyuki Yasuda, Ibaraki (JP); Toshiyasu Sakakura, Ibaraki (JP)

Correspondence Address: NIXON & VANDERHYE, PC, 901 NORTH GLEBE ROAD, 11TH FLOOR, ARLINGTON, VA, 22203, US

(21) Appl. No.: 12/279,116

(22) PCT Filed: Feb. 7, 2007
(86) PCT No.: PCT/JP2007/052099

§ 371 Date: Aug. 12, 2008

(30) Foreign Application Priority Data
Feb. 14, 2006 (JP) 2006-036999
Mar. 23, 2006 (JP) 2006-080016

(51) Int. Cl. C07C 69/78; C07C 41/06

(52) U.S. Cl. 560/107; 568/632

(57) ABSTRACT

Disclosed is a process for production of a carboxylic acid ester from a carboxylic acid and an olefin or production of an ether compound from an alcohol and an olefin at low cost and with high yield in an industrially advantageous manner. The process comprises the step of reacting a carboxylic acid with an olefin to yield a corresponding carboxylic acid ester or reacting an alcohol with an olefin to yield a corresponding ether compound. In the process, a catalyst comprising a combination of (i) at least one metal compound selected from an iron compound, a cobalt compound and a nickel compound and (ii) an acidic compound is used.

### TECHNICAL FIELD

The present invention relates to: a process for production of a carboxylic acid ester by reacting a carboxylic acid with an olefin; and to a process for production of an ether compound by reacting an alcohol with an olefin, each process being performed in the presence of a specific metal compound and a specific acidic compound.

### BACKGROUND ART

There has been known a process for producing a carboxylic acid ester by reacting a carboxylic acid with an olefin.

For example, there is proposed a process for synthesizing an aromatic carboxylic acid ester from norbornene and an aromatic carboxylic acid in the presence of a catalyst made by combining [(Cp*RuCl₂)₂] complex (Cp*: pentamethylcyclopentadienyl group) or silver trifluoromethanesulfonate with an aromatic phosphine ligand (Non-Patent Document 1).

With the process, an aromatic carboxylic acid ester having an exo structure can be obtained with high selectivity. However, the process has disadvantages in that the process requires an expensive catalyst such as ruthenium and silver trifluoromethanesulfonate, the catalyst requires a complex preparation process, and when an aliphatic carboxylic acid is used as a raw material, a reaction does not proceed.

Further, as a process for producing an ether compound by an addition reaction between alcohols and olefins, there is known a process for performing the reaction in the presence of an acidic catalyst.

However, the conventional process has problems in that the process involves an unignorable side reaction, and since a mixture generated from a reaction includes an acidic catalyst, a distilling step that is a separation step in a later stage requires heating, which dissolves an ether compound, resulting in low yield of the ether compound.

Further, there is proposed a process for synthesizing an ether compound from alcohol and norbornene in the presence of a [Cu(Otf)₂] (Cu: copper, Otf: trifluoromethanesulfonate group) complex catalyst (Non-Patent Document 2).

However, in the process, when aliphatic alcohol (isopropanol) is used as a substrate, the yield of an ether compound is insufficient. Further, only a case of using norbornene as an olefin is disclosed, and reaction behavior in a case of other olefins is not suggested at all.

Non-Patent Document 1: Chem. Commun. 2004, p 1620
Non-Patent Document 2: Chem. Commun. 2005, p 5103

### DISCLOSURE OF INVENTION

### Problems to be Solved by the Invention

A first object of the present invention is to provide a process for producing, from a carboxylic acid and an olefin, a corresponding carboxylic acid ester at low cost and with high yield in an industrially advantageous manner, the process having a wide choice of carboxylic acids that are raw materials and being applicable not only to an aromatic carboxylic acid but also to an aliphatic acid.

A second object of the present invention is to provide a process for producing, from an alcohol and an olefin, a corresponding ether compound at low cost and with high yield in an industrially advantageous manner, the process having a wide choice of raw materials and being applicable not only to an aromatic alcohol but also to an aliphatic alcohol, and applicable not only to norbornene as an olefin but also to an aliphatic olefin and an aromatic olefin.

### Means to Solve the Problem

The inventors of the present invention found that a reaction of an aromatic carboxylic acid or an aliphatic carboxylic acid with an olefin in the presence of a specific metal compound and a specific acidic compound effectively yields a corresponding carboxylic acid ester, and that a reaction of an alcohol with an olefin in the presence of a specific metal compound and a specific acidic compound effectively yields a corresponding ether compound, and thus completed the present invention.

The invention of the present application includes the following subject matters.

(1) A process for production of a carboxylic acid ester, including the step of reacting an aliphatic carboxylic acid or an aromatic carboxylic acid with an olefin in the presence of a catalyst including a combination of (i) at least one metal compound selected from an iron compound, a cobalt compound, and a nickel compound and (ii) an acidic compound.

(2) The process according to the item (1), wherein the acidic compound is a Bronsted acid or a metal trifluoromethanesulfonate.

(3) A process for production of an ether compound, including the step of reacting an alcohol with an olefin in the presence of a catalyst including a combination of (i) at least one metal compound selected from an iron compound, a cobalt compound, and a nickel compound and (ii) an acidic compound.

(4) The process according to the item (3), wherein the acidic compound is a Bronsted acid or a metal trifluoromethanesulfonate.

### EFFECT OF THE INVENTION

With the process of the present invention, usage of a catalyst with high handleability that is inexpensive and applicable to wide range of substrates allows reaction of an aliphatic carboxylic acid or an aromatic carboxylic acid with an olefin to yield a corresponding carboxylic ester with high yield, and allows reaction of an alcohol with an olefin to yield a corresponding ether compound with high yield.

### BEST MODE FOR CARRYING OUT THE INVENTION

The present invention relates to a process for production of a carboxylic acid ester and an ether compound with high yields, respectively. At first, the process for production of the carboxylic acid ester is described in details.

In the process for production of the carboxylic acid ester, a reaction of an aliphatic carboxylic acid or an aromatic carboxylic acid with an olefin is carried out in the presence of a catalyst including a combination of (i) at least one metal compound selected from an iron compound, a cobalt compound and a nickel compound and (ii) an acidic compound.

The process for production is represented by the formula below in the case of using a benzoic acid as the carboxylic acid and norbornene as the olefin.

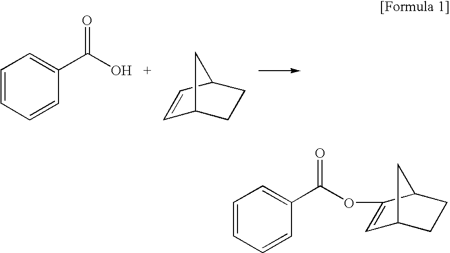

The aliphatic carboxylic acid and the aromatic carboxylic acid used in the esterification reaction are not particularly limited. Examples of the aliphatic carboxylic acid include acetic acid, propionic acid, butyric acid, isobutyric acid, acrylic acid, and methacrylic acid. Examples of the aromatic carboxylic acid include benzoic acid, anisic acid, phenylacetic acid, salicylic acid, o-toluic acid, phthalic acid, isophthalic acid, and terephthalic acid.

Examples of the olefin used in the esterification reaction include, but are not particularly limited to, an aliphatic olefin, a substituted aliphatic olefin, an aromatic olefin, and a substituted aromatic olefin.

Examples of the aliphatic olefin include ethylene, propylene, isopropylene, butene, pentene, hexene, heptene, and octene. Examples of the aromatic olefin include styrene, divinylbenzene, 1-vinylnaphthalene, 2-vinylnaphthalene, and vinylpyridine.

Substituents in the substituted aliphatic olefin and the substituted aromatic olefin are not particularly limited. Examples of the substituents include a phenyl group; a 1-naphthyl group; a 2-naphthyl group; a pyridyl group; a nitro group; an amino group; an amide group; a halogen atom; a carboxyl group; an alkoxy group such as a methoxy group, an ethoxy group, and a phenoxy group; an aralkyl group; and a heterocyclic group. In addition to these olefins, a cyclic olefin can be also used.

Examples of the cyclic olefin include a monocyclic olefin, and a bridged cyclic hydrocarbon represented by a bicyclo compound such as norbornenes which have distortion in the cyclic structure. Examples of the monocyclic olefin include a cyclic olefin with 3-6 carbon atoms such as cyclopropene, cyclobutene, cyclopentene, methylcyclopentene, and cyclohexene. These monocyclic olefins may have no substituent or may have a substituent. Examples of the substituent include an alkyl group and an aryl group.

Norbornenes include, for example, a norbornene derivative represented by formula (2):

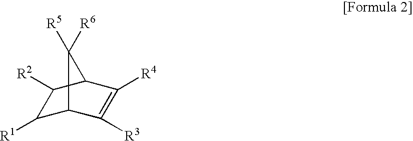

wherein R¹ to R⁶ are independently selected from hydrogen and a lower alkyl group.

Examples of the lower alkyl group include an alkyl group with 1-5 carbon atoms such as a methyl group, an ethyl group, an n-propyl group, an isopropyl group, an i-butyl group, a t-butyl group, an n-butyl group, an n-pentyl group, a neopentyl group, and a t-pentyl group. In terms of high reactivity, a hydrogen atom is especially preferred as the R¹ to R⁶.

Specific examples of the norbornene derivative include norbornene, methylnorbornene, dimethylnorbornene, and ethylnorbornene.

The esterification reaction is carried out in the presence of the catalyst including the combination of (i) at least one metal compound selected from the iron compound, the cobalt compound and the nickel compound and (ii) the acidic compound.

The metal compound is not particularly limited. Examples of the metal compound include FeXₙ(n=2, 3), Fe(CO)₅, Fe₃(CO)₁₂, Fe(CO)₃(EN), Fe(CO)₃(DE), Fe(DE)₂, CpFeX(CO)₂, [CpFe(CO)₂]₂, [Cp*Fe(CO)₂]₂, Fe(acac)₃, Fe(OAc)ₙ (n=2, 3), CoX₂, CO₂(CO)₈, Co(acac)ₙ, (n=2, 3), Co(OAc)₂, CpCO(CO)₂, Cp*Co(CO)₂, NiX₂, Ni(CO)₄, Ni(DE)₂, Ni(acac)₂, and Ni(OAc)₂. An iron compound is preferred as the metal compound. Iron chloride is especially preferred.

X is selected from a hydrogen atom, a halogen atom, a hydroxyl group, a cyano group, an alkoxy group, a carboxylato group, a thiocyanato group. Cp represents a cyclopentadiene group. Acac represents an acetylacetonate group. DE is selected from norbornadiene, 1,5-cyclooctadiene, and 1,5-hexadien. EN is selected from ethylene and cyclooctene. OAc represents an acetate group.

In the reaction of the present invention, it is necessary that not only (i) the metal compound but also (ii) the acidic compound exist as the catalyst in the reaction system.

The acidic compound is not particularly limited. A Bronsted acid and a metal trifluoromethanesulfonate are preferably used. Examples of the Bronsted acid include trifluoromethanesulfonic acid described as follows. Examples of the metal trifluoromethanesulfonate include silver trifluoromethanesulfonate described as follows.

### [Examples of the Bronsted Acids]

### HCl

### H₂SO₄

### CF₃SO₃H

p-[CH₃ (CH₂)₁₁](C₆H₄)SO₃H

### Nafion^{R}

### [Examples of the Metal Trifluoromethanesulfonates]

### Na(OSO₂CF₃)

### Li(OSO₂CF₃)

### Ag(OSO₂CF₃)

### Cu(OSO₂CF₃)₂

### Zn(OSO₂CF₃)₂

### La(OSO₂CF₃)₃

### Sc(OSO₂CF₃)₃

Trifluoromethanesulfonic acid is preferably used as the Bronsted acid. Silver trifluoromethanesulfonate is preferably used as the metal trifluoromethanesulfonate. The amount of the acidic compound is not particularly limited. A molar ratio of the acidic compound to the metal compound ranges from approximately 1/300 to 10, preferably ranges from approximately 1/50 to 3.

The catalyst of the esterification reaction includes the combination of the metal compound and the acidic compound. The metal compound and the acidic compound may be prepared separately and be added to the reaction system. Alternatively, the metal compound may be previously caused to react with the acidic compound outside the reaction system and the resultant may be used as the metal trifluoromethanesulfonate.

A temperature for reaction of the carboxylic acid with the olefin is not particularly limited. The temperature ranges preferably from room temperature to 300° C., more preferably ranges from 60 to 200° C.

The esterification reaction of the present invention may be carried out with a solvent which does not inhibit the esterification reaction, or may be carried out without a solvent. The solvent is not particularly limited, and examples of the solvent include hydrocarbons and ethers. Specific examples of the solvent include benzene, toluene, hexane, tetrahydrofuran, diethyl ether, dibutyl ether, and dioxane.

Secondly, the process for production of the ether compound is described.

In the process for production of the ether compound, etherification reaction of alcohols with olefins is carried out in the presence of a catalyst including a combination of (i) at least one metal compound selected from an iron compound, a cobalt compound, and a nickel compound and (ii) an acidic compound.

The process for production is represented by formula (3) in the case of using phenol as alcohols and norbornene as olefins.

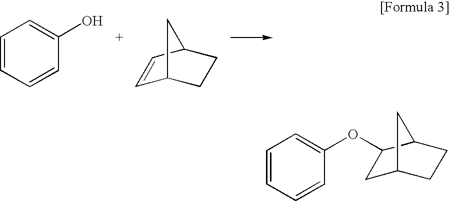

Examples of the alcohols used in the etherification reaction include, but are not particularly limited to, aliphatic alcohols, substituted aliphatic alcohols, aromatic alcohols, and substituted aromatic alcohols. Examples of the aliphatic alcohols include methanol, ethanol, propanol, isopropanol, butanol, pentanol, hexanol, cyclopropanol, cyclopentanol, cyclohexanol, allyl alcohol, ethylene glycol, propylene glycol, butylene glycol, polyglycol and glycerol. Examples of the aromatic alcohols include phenol, naphthol, cresols, xylenols, benzyl alcohols, and phenylethyl alcohols. A substituent in the substituted aliphatic alcohols and the substituted aromatic alcohols is not particularly limited. Examples of the substituent include a phenyl group; a 1-naphthyl group; a 2-naphthyl group; a pyridyl group; a nitro group; an amide group; a halogen atom; a carboxyl group; an alkoxy group such as a methoxy group, an ethoxy group, and a phenoxy group; an aralkyl group; and a heterocyclic group.

The olefins used in the etherification reaction are similar to those described in the above esterification reaction.

In particular, monocyclic olefins are preferably used. Cyclic olefins including bridged cyclic hydrocarbons represented by bicyclo compounds such as norbornenes which have distortion in the cyclic structure are also preferably used.

The etherification reaction is carried out in the presence of the catalyst including the combination of (i) at least one metal compound selected from the iron compound, the cobalt compound and the nickel compound and (ii) the acidic compound.

The metal compound and the acidic compound used in the etherification reaction are not particularly limited. The metal compound and the acidic compound used in the etherification reaction are similar to those described in the above esterification reaction.

The amount of the acidic compound is not particularly limited. A molar ratio of the acidic compound to the metal compound ranges from approximately 1/300 to 10, preferably ranges from approximately 1/50 to 3.

The catalyst in the etherification reaction includes the combination of the metal compound and the acidic compound. The metal compound and the acidic compound may be prepared separately and be added to the reaction system. The metal compound may be previously caused to react with the acidic compound outside the reaction system and the resultant may be used as the metal trifluoromethanesulfonate.

A temperature for reaction of the alcohols with the olefins is not particularly limited. The temperature ranges preferably from room temperature to 300° C., more preferably ranges from 60 to 200° C.

The etherification reaction of the present invention may be carried out with a solvent which does not inhibit the etherification reaction or may be carried out without a solvent. The solvent is not particularly limited, but examples include hydrocarbons and ethers. Specific examples of the solvent include benzene, toluene, hexane, tetrahydrofuran, diethyl ether, dibutyl ether, and dioxane.

### EXAMPLES

The following further details the present invention with reference to Examples.

### (Production of Carboxylic Acid Ester; Examples 1-24 and Comparative Examples 1-4)

### Example 1

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, acrylic acid (20 mmol) and norbornene (20 mmol) were added to the resultant, and the resultant was heated up to 80° C. and caused to react for 18 hours. The reaction mixture was cooled down and then analyzed by a gas chromatography. The yield of acrylic acid norbornyl was 98% by the standard of acrylic acid.

### Example 2

A reaction was carried out as in Example 1 except that, instead of the catalyst obtained by combining a metal compound and an acidic compound, Fe(Otf)₃(III) (Otf: trifluoromethanesulfonate) (0.2 mmol) was used as a metal trifluoromethanesulfonate produced outside the reaction system in advance. The reaction mixture was cooled down and then analyzed by a gas chromatography. The yield of acrylic acid norbornyl was 98% by the standard of acrylic acid.

### Example 3

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, methacrylic acid (20 mmol) and norbornene (20 mmol) were added to the resultant, and the resultant was heated up to 80° C. and caused to react for 18 hours. The reaction mixture was cooled down and then analyzed by a gas chromatography. The yield of acrylic acid norbornyl was 96% by the standard of methacrylic acid.

### Example 4

A reaction was carried out as in Example 3 except that, instead of the catalyst obtained by combining a metal compound and an acidic compound, Fe(Otf)3(III) (0.2 mmol) was used as a metal trifluoromethanesulfonate produced outside the reaction system in advance. The reaction mixture was cooled down and then analyzed by a gas chromatography. The yield of methacrylic acid norbornyl was 96% by the standard of methacrylic acid.

### Example 5

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, acetic acid (20 mmol) and norbornene (20 mmol) were added to the resultant, and the resultant was heated up to 80° C. and caused to react for 18 hours. The reaction mixture was cooled down and then analyzed by a gas chromatography. The yield of acetic acid norbornyl was 98% by the standard of acetic acid.

### Example 6

A reaction was carried out as in Example 5 except that, instead of the catalyst obtained by combining a metal compound and an acidic compound, Fe(Otf)₃(III) (0.2 mmol) was used as a metal trifluoromethanesulfonate produced outside the reaction system in advance. The reaction mixture was cooled down and then analyzed by a gas chromatography. The yield of acetic acid norbornyl was 98% by the standard of acetic acid.

### Example 7

Acetic acid (20 mmol) and cyclohexane (20 mmol) were put in a reaction vessel of 100 ml in capacity with a stirring device, in the presence of Fe(Otf)3(III) (0.2 mmol) as a metal trifluoromethanesulfonate produced outside the reaction system in advance instead of the catalyst obtained by combining a metal compound and an acidic compound. The resultant was heated up to 80° C. and caused to react for 3 hours. The reaction mixture was cooled down and then analyzed by a gas chromatography. The yield of acetic acid cyclohexanyl was 56% by the standard of acetic acid.

### Example 8

Acetic acid (40 mmol) and cyclohexane (20 mmol) were put in a reaction vessel of 100 ml in capacity with a stirring device, in the presence of Fe(Otf)3(III) (0.2 mmol) as the metal trifluoromethanesulfonate produced outside the reaction system in advance instead of the catalyst obtained by combining a metal compound and an acidic compound. A reaction was carried out as in Example 7. The reaction mixture was cooled down and then analyzed by a gas chromatography. The yield of acetic acid cyclohexanyl was 70% by the standard of cyclohexane.

### Example 9

Acetic acid (80 mmol) and cyclohexane (20 mmol) were put in a reaction vessel of 100 ml in capacity with a stirring device, in the presence of Fe(Otf)3(III) (0.2 mmol) as a metal trifluoromethanesulfonate produced outside the reaction system in advance instead of the catalyst obtained by combining a metal compound and an acidic compound. A reaction was carried out as in Example 7. The reaction mixture was cooled down and then analyzed by a gas chromatography. The yield of acetic acid cyclohexanyl was 80% by the standard of cyclohexane.

### Example 10

Acetic acid (80 mmol) and cyclohexane (20 mmol) were put in a reaction vessel of 100 ml in capacity with a stirring device, in the presence of Fe(Otf)3(III) (0.2 mmol) as the metal trifluoromethanesulfonate produced outside the reaction system in advance instead of the catalyst obtained by combining a metal compound and an acidic compound. The resultant was heated up to 80° C. and caused to react for 6 hours. The reaction mixture was cooled down and then analyzed by a gas chromatography. The yield of acetic acid cyclohexanyl was 89% by the standard of cyclohexane.

### Example 11

Acetic acid (80 mmol) and 1-octen (20 mmol) were put in a reaction vessel of 100 ml in capacity with a stirring device, in the presence of Fe(Otf)3(III) (0.2 mmol) as a metal trifluoromethanesulfonate produced outside the reaction system in advance instead of the catalyst obtained by combining a metal compound and an acidic compound. The resultant was heated up to 80° C. and caused to react for 12 hours. The reaction mixture was cooled down and then analyzed by a gas chromatography. The yield of acetic acid octenyl was 78% by the standard of 1-octen.

### Example 12

Fe(Otf)3(III) (0.2 mmol) as a metal trifluoromethanesulfonate produced outside the reaction system in advance instead of the catalyst obtained by combining a metal compound and an acidic compound was put in dibutylether (20 ml) in a reaction vessel of 100 ml in capacity with a stirring device, and then isobutyric acid (20 mmol) and norbornene (20 mmol) were added, and the resultant was heated up to 80° C. and caused to react for 18 hours. The reaction mixture was cooled down and then analyzed by a gas chromatography. The yield of isobutyric acid norbornyl was 99% by the standard of isobutyric acid.

### Example 13

Isobutyric acid (80 mmol) and cyclohexane (20 mmol) were put in a reaction vessel of 100 ml in capacity with a stirring device, in the presence of Fe(Otf)3(III) (0.2 mmol) as a metal trifluoromethanesulfonate produced outside the reaction system in advance instead of the catalyst obtained by combining a metal compound and an acidic compound. The resultant was heated up to 80° C. and caused to react for 24 hours. The reaction mixture was cooled down and then analyzed by a gas chromatography. The yield of isobutyric acid cyclohexanyl was 88% by the standard of cyclohexane.

### Comparative Example 1

Iron chloride (III) (0.2 mmol) was put in dibutylether (20 ml) in a reaction vessel of 100 ml in capacity with a stirring device, and then acrylic acid (20 mmol) and norbornene (20 mmol) were added, and the resultant was heated up to 80° C. and caused to react for 18 hours. The reaction mixture was cooled down and then analyzed by a gas chromatography. The yield of acrylic acid norbornyl was 0% by the standard of acrylic acid.

### Example 14

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, benzoic acid (20 mmol) and norbornene (20 mmol) were added to the resultant, and the resultant was heated up to 80° C. and caused to react for 18 hours. The reaction mixture was cooled down and then analyzed by a gas chromatography. The yield of benzoic acid norbornyl was 99% by the standard of benzoic acid.

### Example 15

A reaction was carried out as in Example 14. The reaction mixture was cooled down and then extracted using a diethylether, and rinsed with a saturated sodium hydrogen carbonate aqueous solution, water, a saturated sodium chloride aqueous solution, and water in this order. The resultant was dried with sodium sulfuric anhydride, and then depressurized and concentrated. The yield of benzoic acid norbornyl was 98% by the standard of benzoic acid.

### Example 16

A reaction was carried out as in Example 14 except that trifluoromethanesulfonic acid (0.6 mmol) was used instead of silver trifluoromethanesulfonate. The reaction mixture was cooled down and then analyzed by a gas chromatography. The yield of benzoic acid norbornyl was 99% by the standard of benzoic acid.

### Example 17

A reaction was carried out as in Example 14 except that, instead of the catalyst obtained by combining a metal compound and an acidic compound, Fe(Otf)3(III) (0.2 mmol) was used as a metal trifluoromethanesulfonate produced outside the reaction system in advance. The reaction mixture was cooled down and then analyzed by a gas chromatography. The yield of benzoic acid norbornyl was 99% by the standard of benzoic acid.

### Comparative Example 2

Iron chloride (III) (0.2 mmol) was put in dibutylether (20 ml) in a reaction vessel of 100 ml in capacity with a stirring device, and then benzoic acid (20 mmol) and norbornene (20 mmol) were added, and the resultant was heated up to 80° C. and caused to react for 18 hours. The reaction mixture was cooled down and then analyzed by a gas chromatography. The yield of benzoic acid norbornyl was 5% by the standard of benzoic acid.

### Comparative Example 3

Silver trifluoromethanesulfonate (0.2 mmol) was put in dibutylether (20 ml) in a reaction vessel of 100 ml in capacity with a stirring device, and then benzoic acid (20 mmol) and norbornene (20 mmol) were added, and the resultant was heated up to 80° C. and caused to react for 18 hours. The reaction mixture was cooled down and then analyzed by a gas chromatography. The yield of benzoic acid norbornyl was 2% by the standard of benzoic acid.

### Comparative Example 4

Trifluoromethanesulfonic acid (0.2 mmol) was put in dibutylether (20 ml) in a reaction vessel of 100 ml in capacity with a stirring device, and then benzoic acid (20 mmol) and norbornene (20 mmol) were added, and the resultant was heated up to 80° C. and caused to react for 18 hours. The reaction mixture was cooled down and then analyzed by a gas chromatography. The yield of benzoic acid norbornyl was 3% by the standard of benzoic acid.

### Example 18

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, benzoic acid (20 mmol) and cyclohexane (20 mmol) were added to the resultant, and the resultant was heated up to 80° C. and caused to react for 18 hours. The reaction mixture was cooled down and then analyzed by a gas chromatography. The yield of benzoic acid cyclohexanyl was 20% by the standard of benzoic acid.

### Example 19

Benzoic acid (40 mmol) and cyclohexane (20 mmol) were put in a reaction vessel of 100 ml in capacity with a stirring device, in the presence of Fe(Otf)3(III) (0.2 mmol) as a metal trifluoromethanesulfonate produced outside the reaction system in advance instead of the catalyst obtained by combining a metal compound and an acidic compound. The resultant was heated up to 80° C. and caused to react for 3 hours. The reaction mixture was cooled down and then analyzed by a gas chromatography. The yield of benzoic acid cyclohexanyl was 73% by the standard of cyclohexane.

### Example 20

A reaction was carried out as in Example 14 except that 1-octen (0.6 mmol) was used instead of norbornene. The reaction mixture was cooled down and then analyzed by a gas chromatography. The yield of benzoic acid-1-octenyl was 20% by the standard of benzoic acid.

### Example 21

A reaction was carried out as in Example 16 except that Co(acac)₃(III) (0.2 mmol) was used instead of iron chloride (III). The reaction mixture was cooled down and then analyzed by a gas chromatography. The yield of benzoic acid norbornyl was 86% by the standard of benzoic acid.

### Example 22

A reaction was carried out as in Example 14 except that cobalt chloride (III) (0.2 mmol) was used instead of iron chloride (III). The reaction mixture was cooled down and then analyzed by a gas chromatography. The yield of benzoic acid norbornyl was 24% by the standard of benzoic acid.

### Example 23

A reaction was carried out as in Example 14 except that nickel chloride (II) (0.2 mmol) was used instead of iron chloride (III). The reaction mixture was cooled down and then analyzed by a gas chromatography. The yield of benzoic acid norbornyl was 12% by the standard of benzoic acid.

### Example 24

Iron chloride (III) (0.2 mmol), silver trifluoromethanesulfonate (0.6 mmol) as a metal trifluoromethanesulfonate, and dibutylether (20 mL) were put in a reaction vessel of 100 ml in capacity with a stirring device, and then the resultant was heated up to 80° C. and caused to react for 2 hours. After cooling the resultant, p-anisic acid (20 mmol) and norbornene (20 mmol) were added to the resultant, and the resultant was heated up to 80° C. and caused to react for 18 hours. The reaction mixture was cooled down and then analyzed by a gas chromatography. The yield of p-anisic acid norbornyl was 99% by the standard of p-anisic acid. (Production of ether compound: Examples 25-38 and Comparative Examples 5 and 6)

### Example 25

Iron chloride (III) (0.2 mmol), silver trifluoromethanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, phenol (20 mmol) and norbornene (20 mmol) were added to the resultant, and the resultant was heated up to 80° C. and caused to react for 18 hours. The reaction mixture was cooled down and then analyzed by a gas chromatography. The yield of phenyl norbornyl ether was 95% by the standard of phenol.

### Example 26

A reaction was carried out as in Example 25. The reaction mixture was cooled down and then extracted using a diethylether, and rinsed with a 10% sodium hydroxide aqueous solution, water, a saturated sodium chloride aqueous solution, and water in this order. The resultant was dried with sodium sulfuric anhydride, and then depressurized and concentrated. The yield of phenyl norbornyl ether was 92% by the standard of phenol.

### Example 27

A reaction was carried out as in Example 25 except that Fe(Otf)3(III) (OTf: trifluoromethanesulfonate group) (0.2 mmol) was used as a metal trifluoromethanesulfonate produced outside the reaction system in advance instead of the catalyst obtained by combining a metal compound and an acidic compound. The reaction mixture was cooled down and then analyzed by a gas chromatography. The yield of phenyl norbornyl ether was 97% by the standard of phenol.

### Example 28

A reaction was carried out as in Example 25 except that trifluoromethanesulfonic acid (0.6 mmol) was used as an acidic compound instead of silver trifluoromethanesulfonate. The reaction mixture was cooled down and then analyzed by a gas chromatography. The yield of phenyl norbornyl ether was 55% by the standard of phenol.

### Comparative Example 5

Iron chloride (III) (0.2 mmol) was put in dibutylether (20 ml) in a reaction vessel of 100 ml in capacity with a stirring device, and then phenol (20 mmol) and norbornene (20 mmol) were added, and the resultant was heated up to 80° C. and caused to react for 18 hours. The reaction mixture was cooled down and then analyzed by a gas chromatography. The yield of phenyl norbornyl ether was 10% by the standard of phenol.

### Comparative Example 6

Silver trifluoromethanesulfonate (0.2 mmol) as metal trifluoromethanesulfonate was put in dibutylether (20 ml) in a reaction vessel of 100 ml in capacity with a stirring device, and then phenol (20 mmol) and norbornene (20 mmol) were added, and the resultant was heated up to 80° C. and caused to react for 18 hours. The reaction mixture was cooled down and then analyzed by a gas chromatography. The yield of phenyl norbornyl ether was 3% by the standard of phenol.

### Example 29

Iron chloride (III) (0.2 mmol), silver trifluoro methanesulfonate (0.6 mmol) as a metal trifluoromethanesulfonate, and dibutylether (20 ml) were put in a reaction vessel of 100 ml in capacity with a stirring device, and then the resultant was heated up to 80° C. and caused to react for 2 hours. After cooling the resultant, methanol (20 mmol) and norbornene (20 mmol) were added to the resultant, and the resultant was heated up to 80° C. and caused to react for 18 hours. The reaction mixture was cooled down and then analyzed by a gas chromatography. The yield of methyl norbornyl ether was 98% by the standard of methanol.

### Example 30

A reaction was carried out as in Example 5 except that Fe(Otf)3(III) (0.2 mmol) was used as a metal trifluoromethanesulfonate produced outside the reaction system instead of the catalyst obtained by combining a metal compound and an acidic compound. The reaction mixture was cooled down and then analyzed by a gas chromatography. The yield of methyl norbornyl ether was 98% by the standard of methanol.

(OTf: trifluoromethanesulfonate group)

### Example 31

A reaction was carried out as in Example 29 except that isopropanol (20 mmol) was used instead of methanol. The reaction mixture was cooled down and then analyzed by a gas chromatography. The yield of isopropyl norbornyl ether was 98% by the standard of isopropanol.

### Example 32

A reaction was carried out as in Example 29 except that allyl alcohol (20 mmol) was used instead of methanol. The reaction mixture was cooled down and then analyzed by a gas chromatography. The yield of allyl norbornyl ether was 92% by the standard of allyl alcohol.

### Example 33

A reaction was carried out as in Example 29 except that benzyl alcohol (20 mmol) was used instead of methanol. The reaction mixture was cooled down and then analyzed by a gas chromatography. The yield of benzyl norbornyl ether was 83% by the standard of benzyl alcohol.

### Example 34

A reaction was carried out as in Example 25 except that 1,3-cyclohexadiene (20 mmol) was used instead of norbornene. The reaction mixture was cooled down and then analyzed by a gas chromatography. The yield of phenyl-2-cyclohexenyl ether was 55% by the standard of phenol.

### Example 35

A reaction was carried out as in Example 25 except that styrene (20 mmol) was used instead of norbornene. The reaction mixture was cooled down and then analyzed by a gas chromatography. The yield of 1-phenylethyl phenyl ether was 31% by the standard of phenol.

### Example 36

A reaction was carried out as in Example 28 except that Fe(acac)₃(III) (0.2 mmol) was used instead of iron chloride (III). The reaction mixture was cooled down and then analyzed by a gas chromatography. The yield of phenyl norbornyl ether was 95% by the standard of phenol.

### Example 37

A reaction was carried out as in Example 28 except that Co(acac)₃(III) (0.2 mmol) was used instead of iron chloride (III). The reaction mixture was cooled down and then analyzed by a gas chromatography. The yield of phenyl norbornyl ether was 92% by the standard of phenol.

### Example 38

A reaction was carried out as in Example 28 except that Ni(acac)₂(II) (0.2 mmol) was used instead of iron chloride (III). The reaction mixture was cooled down and then analyzed by a gas chromatography. The yield of phenyl norbornyl ether was 57% by the standard of phenol.

1. A process for production of a carboxylic acid ester, comprising the step of reacting an aliphatic carboxylic acid or an aromatic carboxylic acid with an olefin in a presence of a catalyst including a combination of (i) at least one metal compound selected from an iron compound, a cobalt compound, and a nickel compound and (ii) an acidic compound.

2. The process according to claim 1, wherein the acidic compound is a Bronsted acid or a metal trifluoromethanesulfonate.

3. A process for production of an ether compound, comprising the step of reacting an alcohol with an olefin in a presence of a catalyst including a combination of (i) at least one metal compound selected from an iron compound, a cobalt compound, and a nickel compound and (ii) an acidic compound.

4. The process according to claim 3, wherein the acidic compound is a Bronsted acid or a metal trifluoromethanesulfonate.
